(19) United States
(12) Patent Application Publication
(10) Pub. No.: US 2009/0016929 A1
(43) Pub. Date: Jan. 15, 2009

(54) ULTRAVIOLET AIR PURIFYING DEVICE

(75) Inventor: John R. Garrett, Vero Beach, FL (US)

Correspondence Address: DOVAS LAW P.C., 307 BAINBRIDGE STREET, PHILADELPHIA, PA, 19147, US

(73) Assignee: AirGorilla Corporation, Vero Beach, FL (US)

(21) Appl. No.: 12/172,223

(22) Filed: Jul. 12, 2008

Related U.S. Application Data
(60) Provisional application No. 60/949,425, filed on Jul. 12, 2007.

(51) Int. Cl. A61L 9/20

(52) U.S. Cl. 422/4; 422/121

(57) ABSTRACT

An air purifying device is provided. The air purifying device includes a mounting body including a magnet configured for connection to at least one of a duct and a plenum of an air handling system. An ultraviolet lamp is connected to and extends from the mounting body. The present invention further provides a method for purifying air and disinfecting surfaces within an air handling system.

### CROSS REFERENCE TO RELATED APPLICATION(S)

This application claims the benefit of U.S. Provisional Patent Application No. 60/949,425, filed Jul. 12, 2007, which is incorporated by reference as if fully set forth.

### BACKGROUND

The air within our living and working environments may include several types of harmful contaminates including particles, organic and inorganic gases, and bioaerosols such as bacteria, viruses and molds. Ultraviolet air purifiers may be used to remove some of these contaminates from the air. Such devices typically use ultraviolet light to convert contaminates into less harmful or completely benign substances.

Known ultraviolet air purifiers may be used in an enclosed environment to provide effective air purification to promote the health of building occupants. These known devices are often difficult to install requiring special tools or special skills. Moreover, ultraviolet air purifiers include ultraviolet lamps which must be periodically replaced. Known devices often require tools or special skills for lamp replacement making such replacement especially inconvenient.

In view of the above, it would be desirable to provide an ultraviolet air purifying device which can be easily integrated with existing air handling systems to provide effective air purification. Such system should allow for fast and convenient lamp replacement without the use of tools or special skills. Such system should also be easily integrateable with new air handling systems using conventional methods of air handling system design.

### SUMMARY

The present invention provides an air purifying device. The air purifying device includes a mounting body including a magnet configured for connection to at least one of a duct and a plenum of an air handling system. An ultraviolet lamp is connected to and extends from the mounting body.

The present invention further provides a method for purifying air and disinfecting surfaces within an air handling system. The method includes providing an air duct or air plenum which defines an air flow path of the air handling system. An air purifying device is provided including a mounting body having a magnet and including an ultraviolet lamp connected to and extending from the mounting body. The mounting body is magnetically connected to the duct or plenum. The ultraviolet lamp is positioned in the duct or plenum within the air flow path of the air handling system, and the ultraviolet lamp is illuminated.

### BRIEF DESCRIPTION OF THE DRAWING(S)

The foregoing Summary as well as the following detailed description will be readily understood in conjunction with the appended drawings which illustrate preferred embodiments of the invention. In the drawings:

FIG. 1 is a rear perspective view of an air purifying device according to a first preferred embodiment of the present invention.

FIG. 2 is a front perspective view of the air purifying device of FIG. 1.

FIG. 3 is a front elevation view of the air purifying device of FIG. 1.

FIG. 4A is a rear elevation view of the air purifying device of FIG. 1.

FIG. 4B is a cross-section view of the air purifying device of FIG. 1 taken along line 4B-4B of FIG. 4A.

FIG. 5 is a front perspective view of the air purifying device of FIG. 1 and an air duct during a process of installing the air purifying device in the air duct.

FIG. 6 is a side elevation cross-section view of the air purifying device of FIG. 1 installed in the air duct of FIG. 5.

FIG. 7 is a front perspective view of the air purifying device of FIG. 1, a magnetizable adapter, and an air duct during a process of installing the air purifying device in the air duct.

FIG. 8 is a side elevation cross-section view of the air purifying device of FIG. 1 installed with the magnetizable adapter of FIG. 7 in the air duct of FIG. 7.

FIG. 9A is a rear elevation view of a magnetic adapter for use with the air purifying device of FIG. 1.

FIG. 9B is a side elevation view of the magnetic adapter of FIG. 9A taken along line 9B-9B of FIG. 9A.

FIG. 9C is a front elevation view of the magnetic adapter of FIG. 9A taken along line 9C-9C of FIG. 9B.

FIG. 10 is an exploded rear perspective view of an assembly including the air purifying device of FIG. 1 and the magnetic adapter of FIG. 9A.

FIG. 11 is a side elevation view of the assembly including the air purifying device of FIG. 1 and the magnetic adapter of FIG. 9A.

FIG. 12 is a front perspective view of an air purifying device according to a second preferred embodiment of the present invention.

FIG. 13 is a rear perspective view of the air purifying device of FIG. 12.

FIG. 14 is a front perspective view of an assembly including the air purifying device of FIG. 12 and a magnetizeable adapter.

FIG. 15 is a front perspective view of an air purifying device according to a third preferred embodiment of the present invention.

FIG. 16 is a diagram showing a method of purifying air according to a preferred embodiment of the present invention

### DETAILED DESCRIPTION OF THE PREFERRED EMBODIMENT(S)

Certain terminology is used in the following description for convenience only and is not limiting. The words “right,” “left,” “top,” and “bottom” designate directions in the drawings to which reference is made. The words “a” and “one” are defined as including one or more of the referenced item unless specifically stated otherwise. This terminology includes the words above specifically mentioned, derivatives thereof, and words of similar import. The phrase “at least one” followed by a list of two or more items, such as A, B, or C, means any individual one of A, B or C as well as any combination thereof.

The preferred embodiments of the present invention are described below with reference to the drawing figures where like numerals represent like elements throughout.

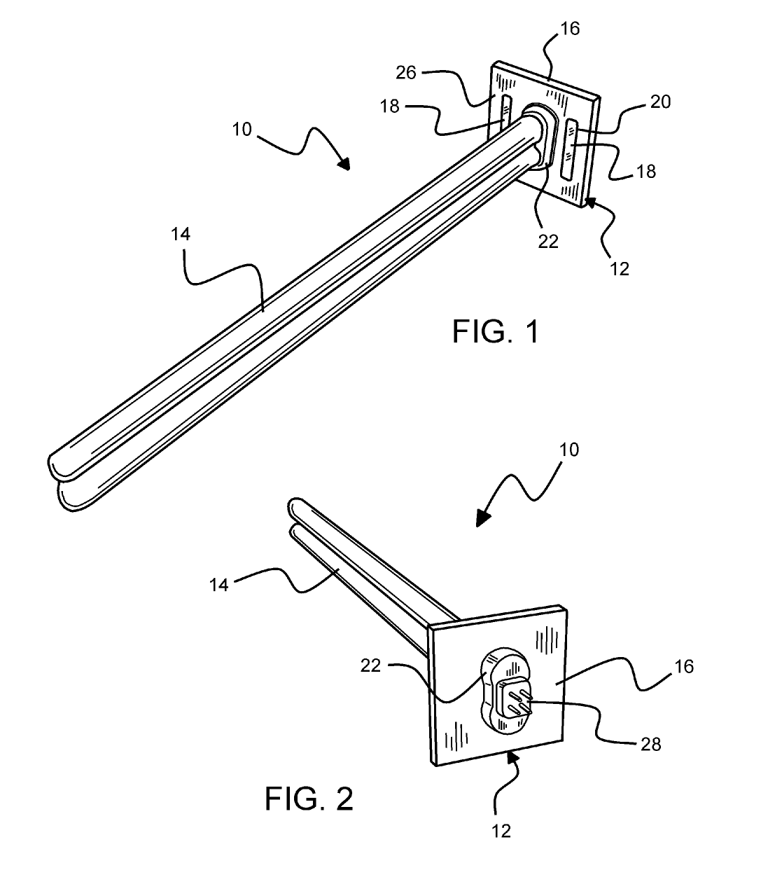

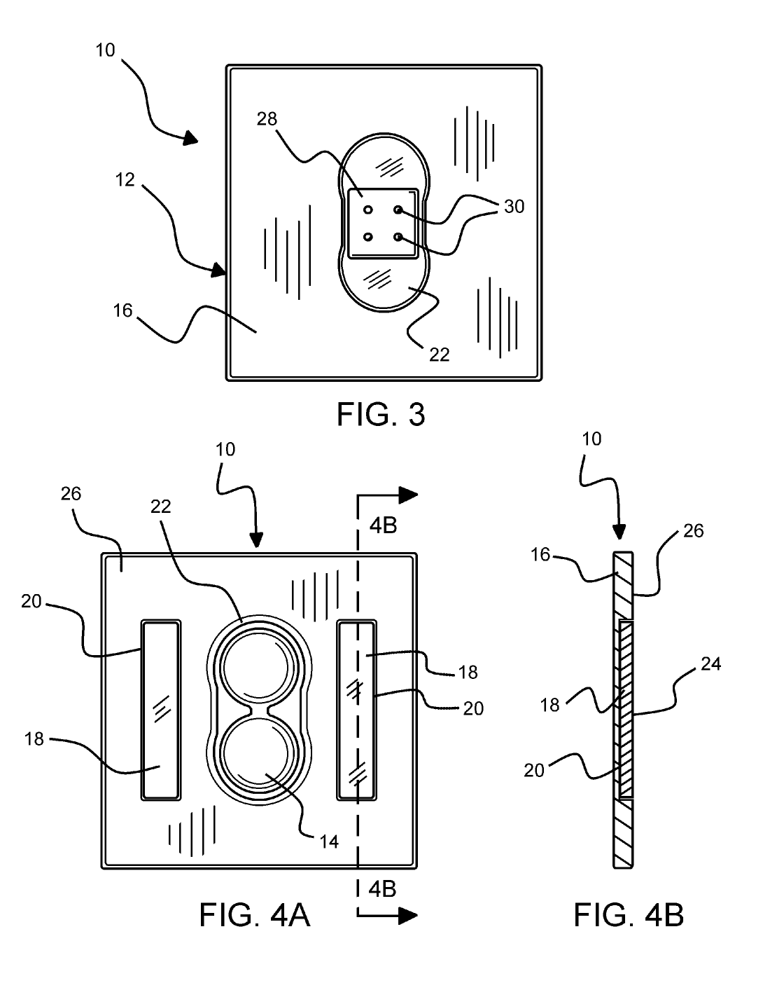

Referring to FIGS. 1-6, an air purifying device 10 according to a first preferred embodiment of the present invention is shown. The air purifying device 10 is configured for connection to an air duct or air plenum of an air handling system such as a residential or commercial HVAC system. By this specification, it is intended that any air passage in an air handling system would be categorized as either an air duct or air plenum, such that the air purifying device 10 may be connected on any suitable portion of an air handling system.

The air purifying device 10 includes a mounting body 12 and an ultraviolet lamp 14 connected to and extending from the mounting body 12. The mounting body 12 preferably includes a mounting plate 16 and permanent magnets 18 attached to the mounting plate 16. The permanent magnets 18 are attached within recesses 20 on the mounting plate 16. The ultraviolet lamp 14 is attached within a protuberance 22 of the mounting plate 16, such protuberance 22 providing structural strength to prevent breakage of the ultraviolet lamp 14 from handling or from the passage of high speed air flow. A connector housing 28 includes connector pins 30 connected to the ultraviolet lamp 14. The connector pins 30 are configured for connection to a separately provided ballast and power supply for providing power to the ultraviolet lamp 14 to illuminate the ultraviolet lamp 14. Alternatively, a ballast or power supply may be provided attached to or integral with the mounting body 12.

The permanent magnets 18 are preferably metallic and are of suitable magnetic field strength to firmly secure the air purifying device 10 to a magnetizable material, wherein within this specification, a magnetizable material is defined as a material which exhibits ferromagnetism or ferrimagnetism. Alternatively, the permanent magnets 18 may be of composite construction including one or more of a polymeric material, a ceramic material, and a metallic material, or alternatively, any other suitable material. The permanent magnets 18 are preferably attached within the recesses 20 via an adhesive, or alternatively, via screws or magnetization. A mounting surface 24 of each of the permanent magnets 18 is preferably generally coplanar with a mounting surface 26 of the mounting plate 16 to permit the mounting surface 26 of the mounting plate 16 to lay flush against a magnetizable surface on which the air purifying device 10 is attached.

The mounting plate 16, including the protuberence 22 and connector housing 28, is preferably formed as a single part from polymeric material, and more preferably, the mounting plate 16 is formed of ABS (acrylonitrile butadiene styrene) or polycarbonate. Alternatively, ceramic or other suitable materials may be used to form the mounting plate 16. While the mounting plate 22 shown in FIGS. 1-4 is generally low-profile and includes a substantially flat mounting surface 26, alternatively, a mounting plate may be provided including curved or angled surfaces and having any suitable shape, body thickness, or particular surface features as required for its application. The ultraviolet lamp 14 is preferably permanently adhered within the protuberance 22 such that the air purifying device 10 is provided as a generally non-serviceable single piece unit.

When powered, the ultraviolet lamp 14 preferably emits relatively intense radiation at a wavelength of about 254 nm, which corresponds to the wavelength commonly known as germicidal. The ultraviolet lamp 14 may alternatively emit relatively intense radiation across a broad spectrum of wavelengths between about 200 nm and 300 nm. Alternatively, the ultraviolet lamp 14 may further emit radiation at ozone-generating wavelengths including wavelengths less than 200 nm, such as an ideal ozone-generating wavelength of about 180 nanometers. While a single ultra violet lamp 14 is shown, alternatively, two or more ultraviolet lamps having the same or dissimilar radiation emission qualities may be connected to a mounting body of the air purifying device.

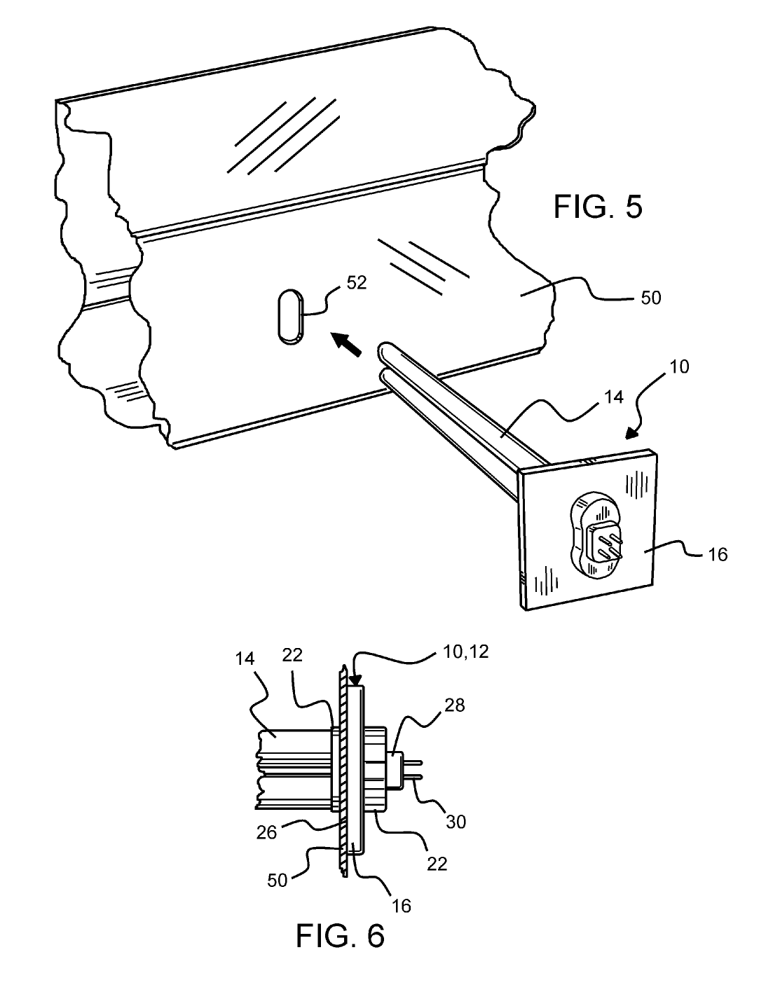

Referring to FIGS. 5 and 6, an air duct 50 is shown having an aperture 52 through which the ultraviolet lamp 14 may be inserted during installation of the air purifying device 10 on the air duct 50. One skilled in the art will readily understand that the air duct 50 may be representative of any portion of an air handling system, such as a residential or commercial HVAC system. The aperture 52 may be formed after the installation of the duct 50, such as instances during which the air purifying device 10 is retrofitted to an existing air handling system. Alternatively, the aperture 52 may be present in the air duct 50 prior to installation of the air duct 50 in an air handling system. While the aperture 52 is shown as a rounded slot, the aperture 52 may alternatively be formed in any suitable shape including circular, rectangular, elliptical or irregularly shaped. The aperture 52 may be any size suitable to fit the ultraviolet lamp 14 therethrough.

During installation of the air purifying device 10 on the air duct 50, the ultraviolet lamp 14 passes through the aperture 52 into an air flow path, and the permanent magnets 18 are magnetically connected to the duct 50. If the surface of the duct 50 on which the air purifying device 10 is mounted is generally parallel to the air flow path, the ultraviolet lamp 14 will extend generally perpendicular to the air flow path. When the air purifying device 10 is installed on the air duct 50, the mounting surface 26 of the mounting plate 16 is attached substantially flush with an outer surface of the air duct 50, as shown in FIG. 6, to permit only a limited amount of air to escape through the aperture 52 during operation of the air handling system. Alternatively, gaskets or sealants may also be provided between the mounting plate 16 and duct 50 to minimize air leakage.

When the ultraviolet lamp 14 is illuminated within the air duct 50, air flowing along the air flow path, or generally stationary air if the air handling system is inactive, is purified. The extent to which air is purified may depend on the rate of flow of air along the air flow path, the relative size of the duct and the ultraviolet lamp 14, the power of the ultraviolet lamp 14, and the particular wavelength or wavelengths at which the ultraviolet lamp 14 operates. When illuminated, the ultraviolet lamp 14 is further effective in disinfecting inner surfaces of the duct 50 which are in relatively close proximity to the ultraviolet lamp 14.

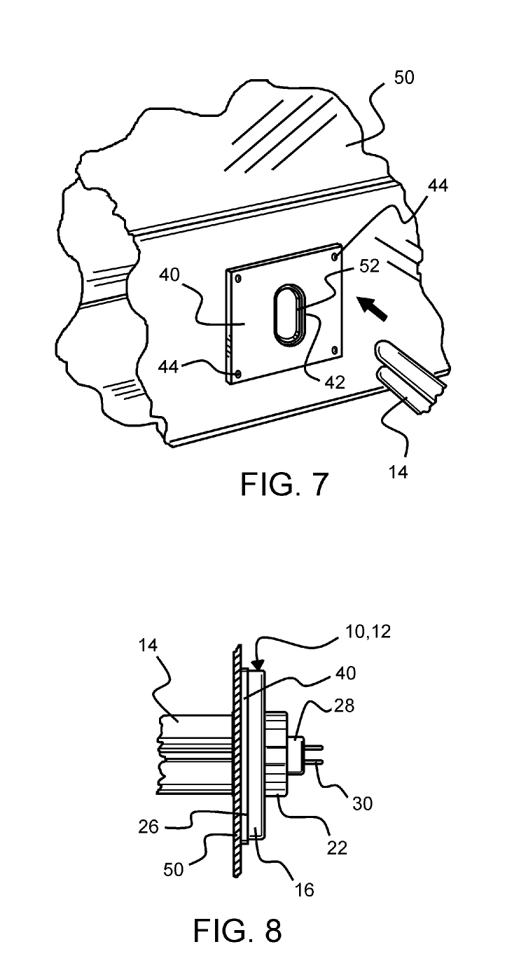

Referring to FIGS. 7 and 8, in instances where the air duct 50 is not magnetizeable or insufficiently magnetizeable, such as in the case when the duct 50 is formed of stainless steel, aluminum or non-metallic material, a low profile magnetizable adapter 40 may be attached to the air duct 50 to permit magnetic attachment of the air purifying device 10 thereto. The magnetizable adapter 40 is preferably constructed of magnetizable sheet metal, or alternatively, of any suitable low profile magnetizable material. The magnetizable adapter 40 includes an aperture 42 which during installation is aligned with the aperture 52 on the air duct 50 to permit passage of the ultraviolet lamp 14 therethrough into an air flow path of the air duct 50. Fasteners 44 secure the magnetizable adapter 40 to the air duct 50. Alternatively, the magnetizable adapter 40 may be attached to the air duct 50 using an adhesive or in any other suitable manner. When the air purifying device 10 is installed, the mounting surface 26 of the mounting plate 16 is attached substantially flush with an outer surface of the magnetizable adapter 40, as shown in FIG. 8, to permit only a limited amount of air to escape during operation of the air handling system.

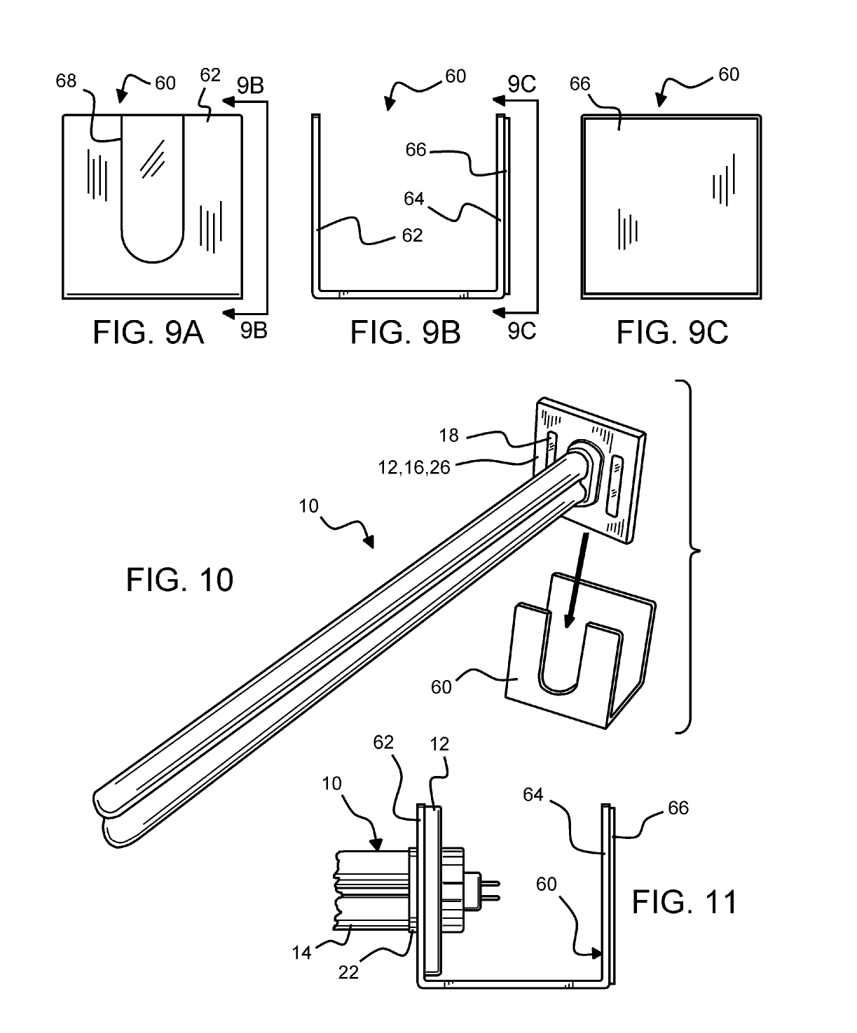

Referring to FIGS. 9A-9C, 10 and 11, a magnetic adapter 60 for use with the air purifying device 10 is shown. The magnetic adapter 60 includes a first arm 62 and a second arm 64. The second arm 64 includes a permanent magnet 66 attached thereto. The first arm includes an aperture 68. As an alternative to providing the permanent magnet 66 separately attached to the second arm 64 as shown, the magnetic adapter 60 may be permanently magnetized. Using the magnetic adapter 60, the air purifying device 10 may be installed in an air duct or plenum without providing an aperture in the duct or plenum. The air purifying device 10 may be attached to the first arm 62 of the magnetic adapter 60 with the ultraviolet lamp 14 and protuberance 22 protruding through the aperture 68 as shown in FIGS. 10 and 11. The second arm 64 maybe attached via the permanent magnet 66 to an inner surface of an air duct or air plenum with the ultraviolet lamp extending generally perpendicularly into an air flow path. If necessary, a relatively small aperture may be formed in the duct or plenum to permit passage of a line for providing power to the ultraviolet lamp 14 via the connector pins 30. While the magnetic adapter 60 is shown as generally U-shaped, alternatively, a magnetic adapter may be provided in any suitable shape or form to permit connection to the air purifying device 10 and the inner surface of an air duct or air plenum.

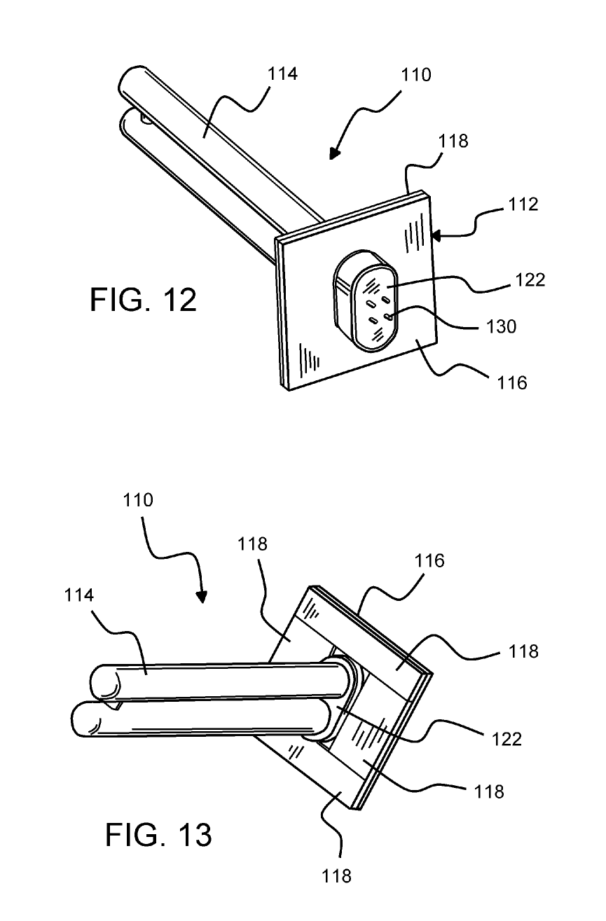

Referring to FIGS. 12 and 13, an air purifying device 110 according to a second preferred embodiment of the present invention is shown. The air purifying device 110 includes a mounting body 112 and an ultraviolet lamp 114 connected to and extending from the mounting body 112. The mounting body 112 includes a mounting plate 116 and permanent magnets 118 in the form of sheets attached to the mounting plate 16. The permanent magnets 118 are attached on a surface of the mounting plate 116, surrounding the ultraviolet lamp 114, providing a substantially continuous magnetic surface, as shown in FIG. 13. The ultraviolet lamp 114 is attached within a protuberance 122 of the mounting plate 116, such protuberance 122 providing structural strength to prevent breakage of the ultraviolet lamp 114 from handling or from the passage of high speed air flow. Connector pins 130 extend from the protuberance 122 and are connected to the ultraviolet lamp 114. The connector pins 130 are configured for connection to a separately provided ballast and power supply for providing power to the ultraviolet lamp 114 to illuminate the ultraviolet lamp 114. When the air purifying device 110 is installed on an air duct or plenum, the permanent magnets 118 are attached substantially flush with an outer surface of the air duct or air plenum to permit only a limited amount of air to escape when air passes through the duct or plenum.

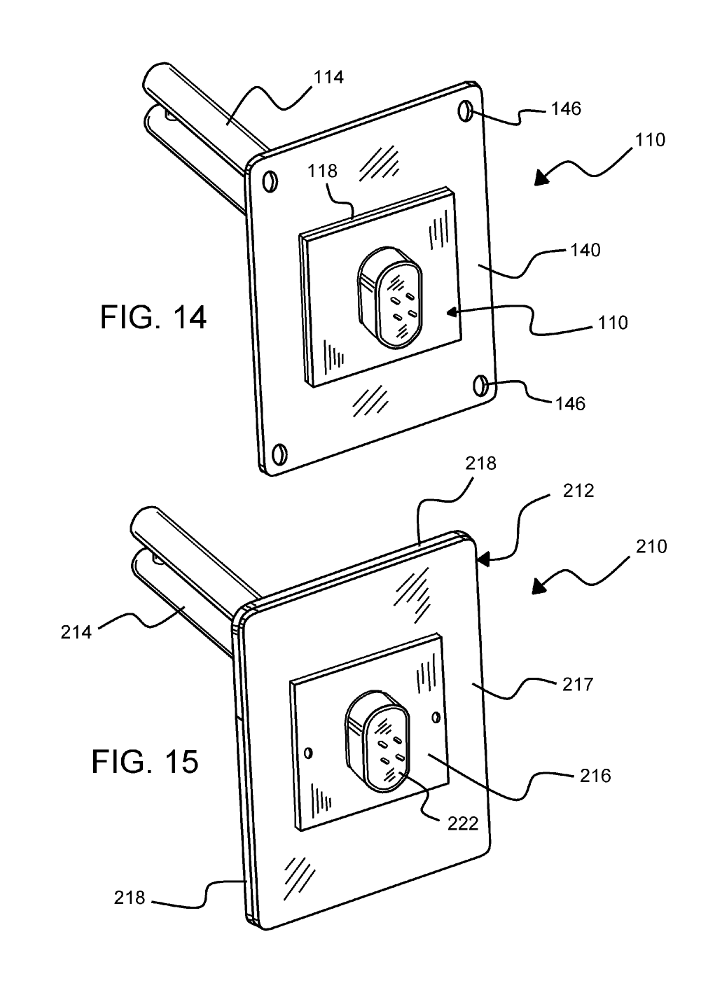

Referring to FIG. 14, in instances where an air duct or air plenum is not magnetizeable or insufficiently magnetizeable, a low profile magnetizable adapter 140 may be attached to the air duct or plenum to permit magnetic attachment of the air purifying device 110 thereto. The magnetizable adapter 140 includes an aperture which during installation is aligned with an aperture in an air duct or plenum, in the manner shown in FIG. 7 with reference to the magnetizable adapter 40, to permit passage of the ultraviolet lamp 14 therethrough into an air flow path of the air duct or plenum. Mounting apertures 146 permit the magnetizable adapter 140 to be mechanically fastened to an air duct or plenum. The permanent magnets 118 are magnetically attached substantially flush with an outer surface of the magnetizable adapter 140, as shown in FIG. 14, to permit only a limited amount of air to escape when air passes through the air duct or plenum.

Referring to FIG. 15, an air purifying device 210 according to a third preferred embodiment of the present invention is shown. The air purifying device 210 includes a mounting body 212 and an ultraviolet lamp 214 connected to and extending from the mounting body 212. The mounting body 212 includes a first mounting plate 216, a second mounting plate 217 connected to the first mounting plate 216, and permanent magnets 218 attached to the mounting plate 217. The permanent magnets 218 are attached on a surface of the mounting plate 217, surrounding the ultraviolet lamp 214 providing a substantially continuous magnetic surface. The ultraviolet lamp 214 is attached within a protuberance 222 of the mounting plate 216. When the air purifying device 210 is installed in an air duct or plenum, the surface of the permanent magnets 218 are magnetically attached substantially flush with an outer surface of the air duct or plenum to permit only a limited amount of air to escape when air passes through the air duct or plenum.

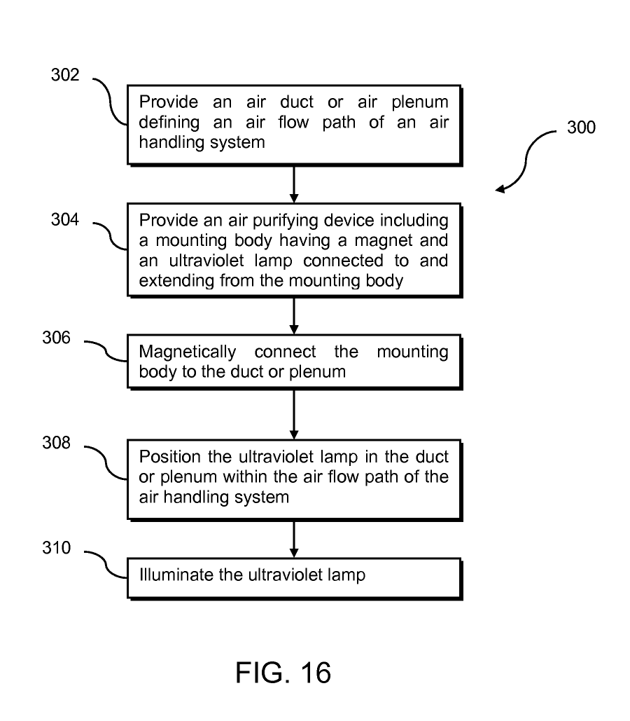

Referring to FIG. 16, a diagram showing a method 300 for purifying air and disinfecting surfaces within an air handling system is shown. The method 300 includes providing an air duct or air plenum defining an air flow path of the air handling system (step 302). An air purifying device is provided including a mounting body having a magnet and including an ultraviolet lamp connected to and extending from the mounting body (step 304). The mounting body is magnetically connected to the duct or plenum (step 306). The ultraviolet lamp is positioned in the duct or plenum within the air flow path of the air handling system (step 308), and the ultraviolet lamp is illuminated (step 310). Preferably, a flow of air is provided through the air flow path over the ultraviolet lamp.

While the preferred embodiments of the invention have been described in detail above, the invention is not limited to the specific embodiments described above, which should be considered as merely exemplary. Further modifications and extensions of the present invention may be developed, and all such modifications are deemed to be within the scope of the present invention as defined by the appended claims.

What is claimed is:

1. An air purifying device comprising:
a mounting body comprising at least one magnet, the mounting body configured for connection to at least one of a duct and a plenum of an air handling system; and
at least one ultraviolet lamp connected to and extending from the mounting body.

2. The air purifying device of claim 1, wherein the mounting body comprises a mounting plate and at least one permanent magnet connected to the mounting plate.

3. The air purifying device of claim 2, wherein the at least one permanent magnet comprises at least one permanent sheet magnet connected to a surface of the mounting plate.

4. The air purifying device of claim 3, wherein the at least one ultraviolet lamp is attached to the mounting plate distanced from an edge of the mounting plate generally surrounded by the at least one sheet magnet.

5. The air purifying device of claim 2, wherein the mounting plate comprises at least one recess, and the at least one permanent magnet is connected in the at least one recess.

6. The air purifying device of claim 5, wherein the mounting plate includes a mounting surface, and the at least one permanent magnet includes a mounting surface, and wherein the mounting surface of the mounting plate and the mounting surface of the at least one permanent magnet are generally coplanar.

7. The air purifying device of claim 2, wherein the mounting plate comprises ABS.

8. The air purifying device of claim 1, wherein the mounting body comprises a polymeric mounting plate and at least one permanent magnet connected to the polymeric mounting plate.

9. The air purifying device of claim 1, wherein the mounting body comprises a mounting plate including a mounting surface, and wherein the at least one ultraviolet lamp comprises an elongated ultraviolet lamp which extends approximately perpendicular from the mounting surface of the mounting plate.

10. The air purifying device of claim 1, further comprising a low profile magnetizable adapter which is removably magnetically connectable to the mounting body, the low profile magnetizable adapter comprising an opening for receiving therethrough the at least one ultraviolet lamp, wherein the low profile magnetizable adapter is configured for connection to a generally non-magnetizable material.

11. The air purifying device of claim 1, further comprising a magnetic adapter comprising:
a first arm removably magnetically connectable to the mounting body and comprising an opening for receiving the at least one ultraviolet lamp upon connection to the mounting body; and
a second arm, generally opposite the first arm, comprising a permanent magnet.

12. The air purifying device of claim 1, wherein the at least one ultraviolet lamp emits at least a wavelength of radiation of about 254 nm.

13. A method for purifying air and disinfecting surfaces within an air handling system comprising:
providing at least one of a duct and a plenum defining an air flow path of the air handling system;
providing an air purifying device comprising:
a mounting body comprising at least one magnet; and
at least one ultraviolet lamp connected to and extending from the mounting body; and
magnetically connecting the mounting body to the at least one of the duct and the plenum and positioning the at least one ultraviolet lamp in the at least one of the duct and the plenum within the air flow path of the air handling system; and
illuminating the at least one ultraviolet lamp.

14. The method of claim 13, further comprising:
providing the at least one ultraviolet lamp as at least one elongated ultraviolet lamp; and
positioning the at least one elongated ultraviolet lamp approximately perpendicular with the flow path of the air handling system.

15. The method of claim 14, further comprising inserting the at least one elongated ultraviolet lamp through an aperture of the at least one of the duct and the plenum of the air handling system; and
magnetically connecting the mounting body to the at least one of the duct and the plenum.

16. The method of claim 13, further comprising:
providing the at least one of the duct and the plenum with at least one aperture;
inserting the at least one ultraviolet lamp through the at least one aperture of the at least one of the duct and the plenum of the air handling system; and
magnetically connecting the mounting body to the at least one of the duct and the plenum.

17. The method of claim 13, further comprising emitting from the at least one ultraviolet lamp a broad spectrum of radiation including a plurality of wavelengths between about 200 nm and 300 nm.

18. The method of claim 13, further comprising:
providing a magnetizable adapter;
connecting the magnetizable adapter to the at least one of the duct and the plenum; and
magnetically connecting the mounting body to the magnetizable adapter.

19. The method of claim 13, further comprising:
providing a magnetic adapter;
magnetically connecting the mounting body of the air purifying device to the magnetic adapter; and
connecting the magnetic adapter to an inner surface of the at least one of the duct and the plenum of the air handling system.

20. The method of claim 13, further comprising:
providing a magnetic adapter comprising a first arm and a second arm connected to the first arm generally opposite to the first arm;
magnetically connecting the mounting body of the air purifying device to the first arm; and
magnetically connecting the second arm to an inner surface of the at least one of the duct and the plenum of the air handling system.

21. The method of claim 13, further comprising:
providing a magnetic adapter comprising a first arm and a second arm connected to the first arm generally opposite to the first arm;
magnetically connecting the mounting body of the air purifying device to the first arm; and
connecting the second arm to an inner surface of the at least one of the duct and the plenum of the air handling system.

22. The method of claim 13, further comprising providing a flow of air through the air flow path of the air handling system.
